(19) United States
(12) Patent Application Publication
(10) Pub. No.: US 2007/0042454 A1
(43) Pub. Date: Feb. 22, 2007

(54) Device and method of detecting streptococcal mutans

(75) Inventor: Paul Nix, Jackson, NJ (US)

Correspondence Address: HOFFMANN & BARON, LLP, 6900 JERICHO TURNPIKE, SYOSSET, NY, 11791, US

(73) Assignee: Princeton Separations, Inc.

(21) Appl. No.: 11/206,316

(22) Filed: Aug. 17, 2005

(51) Int. Cl. C12Q 1/04; C12M 3/00

(52) U.S. Cl. 435/34; 435/287.1

(57) ABSTRACT

A device, kit and method for detecting oral bacteria are provided. The device is a tube, which includes on its inside surface a coating of an agar medium selective for growing gram-positive bacteria. The device is particularly suitable for detecting the extent of growth of mutans streptococci, which provides an indication of the susceptibility of a patient to dental caries.

### FIELD OF THE INVENTION

The present invention relates to devices and methods for detecting gram positive bacteria, which are present in an oral cavity. In particular, the invention relates to a cell culture tube, which includes on its inside surface a selective agar medium for growing gram positive oral bacteria, such as mutans streptococci.

### BACKGROUND OF THE INVENTION

Mutans streptococci are considered to be the primary bacterial cause of dental caries. The extent of growth of the mutans streptococci provides an indication of the susceptibility of a patient to dental caries.

Methods are known for detecting and quantifying mutans streptococci in the saliva of a patient. For example, one method involves collecting a sample of saliva on the surface of a solid plastic strip; introducing the sample on the plastic strip into a liquid medium which is selective for mutans streptococci; incubating in the liquid medium to allow growth of visible colonies; and detecting and counting colonies of mutans streptococci on the surface of the plastic strip. A disadvantage of this method is that it requires an incubation of 48 hours before the plastic strips can be evaluated for colonies. A further disadvantage is that the sample is a saliva sample. Therefore, the sample is not taken directly from the teeth surfaces, where mutans streptococci reside as a major component of dental plaques.

Another method involves growing mutans streptococci on a solid agar medium selective for mutans streptococci. The agar medium employed is a modified Mitis-Salivarius agar medium. This prior method involves having a patient chew paraffin wax to stimulate saliva, removing the paraffin wax from the mouth; introducing the stimulated saliva from the patient into a diluent solution; and contacting the diluent solution containing the saliva sample with an agar medium-coated paddle. This prior method further involves incubating the coated paddle which had been placed in contact with the sample; and thereafter semi-quantitatively determining the amount of mutans streptococci in the test saliva by determining the colony density on the paddle. A disadvantage of this method is that it involves an incubation of 48 hours before the colony density on the paddle can be evaluated. Another disadvantage is that the colonies are very difficult to read and interpret due to the opaque characteristics of the paddle. Yet another disadvantage is that it is invasive and time consuming for the patient. For example, the patient is asked to chew paraffin wax for several minutes during which time saliva needs to be collected at regular time intervals.

In view of the foregoing, there is a need in the art for further devices, kits and methods for detecting oral bacteria indicative of dental caries. In particular, it would be desirable if the device permitted the bacterial colonies to be easily seen and counted. Moreover, it would be desirable if the method required shorter incubation times, and was less invasive and time consuming for the patient. It would also be desirable if the device and method could be used by dentists and dental health professionals in their office, as opposed to a laboratory setting, to provide for the determination of mutans streptococci.

### SUMMARY OF THE INVENTION

The present invention provides a device, kit and method for the determination of the extent of growth of gram positive bacteria in an oral cavity of a patient. The extent of growth provides an indication of the susceptibility of the patient to dental caries. The device, kit and method are particularly suitable for the determination of mutans streptococci by dental professionals in a dentist's office.

In particular, the invention provides a device for detecting oral bacteria that includes a generally cylindrical tube having an open end for receiving a sample and an opposed closed end. The tube includes an inner surface and an opposed outer surface, the inner surface being coated with an agar coating selective for gram positive bacteria. The agar coating is particularly useful for inducing the growth of mutans streptococci.

The present invention also provides a cell culture tube including an inner surface substantially coated with a composition selective for oral bacteria. This coating composition includes Mitis Salivarius agar enriched with sucrose, the sucrose being present in an amount of about 10 to about 20% (w/v).

Further provided is a sterile liquid transfer media suitable for transporting bacteria into the cell culture tube of the present invention. The transfer media is a liquid bacterial transport formulation protective of oral bacteria. The media composition includes at least one peptone in amount of about 0.1% to about 0.5% (w/v); at least one saccharide in an amount of about 0.1 to about 0.5% (w/v); and at least one polyol in an amount of about 0.1 to about 0.25% (w/v).

Also provided is a kit for detecting oral bacteria. The kit includes a generally cylindrical tube having an open end for receiving a sample and an opposed closed end, the tube including an inner surface and an opposed outer surface, the inner surface being coated with an agar coating selective for oral bacteria. The kit also includes a cap for closing the open end of the tube; and liquid media. The liquid media may be inoculated with a sample for transfer into the coated tube.

The present invention also provides a method for detecting oral bacteria. This method includes providing an oral sample from a tooth surface. The method further includes providing a generally cylindrical tube having an open end and an opposed closed end, the tube including an inner surface and an opposed outer surface, the inner surface being coated with an agar coating selective for oral bacteria. The method also involves inoculating liquid media with the oral sample; transferring the inoculated media into the tube; removing the inoculated media from the tube; and subsequently incubating the tube until bacterial colonies are detectable on the agar coating.

### BRIEF DESCRIPTION OF THE DRAWINGS

FIG. 1 is a schematic, perspective view of one embodiment of the device of the present invention.

FIG. 1A is a cross-sectional view of the device of FIG. 1, taken along line 1A.

FIG. 2 is a schematic, perspective view of a cap, which may be used to close the open end of the device of FIG. 1.

FIG. 2A is a top view of the cap of FIG. 2.

FIG. 3 is a schematic, perspective view of the components of one embodiment of a test kit of the present invention.

FIG. 4 is a schematic, perspective view of a coated cell culture tube of the present invention after it has been brought in contact with a patient's oral specimen and thereafter incubated overnight at 37° C.

### DETAILED WRITTEN DESCRIPTION

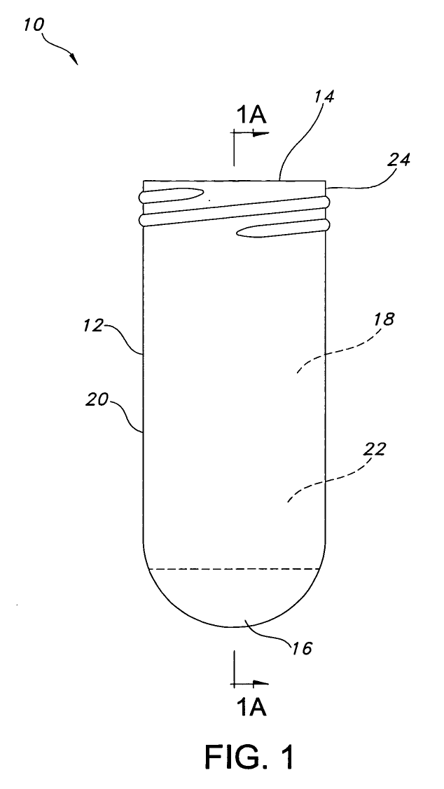

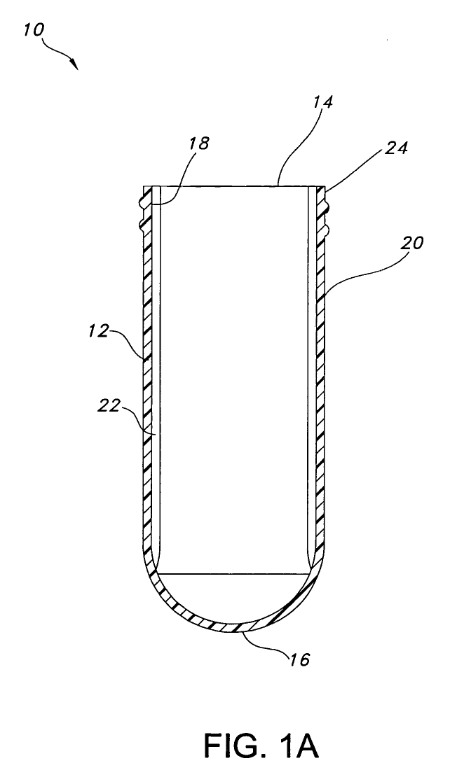

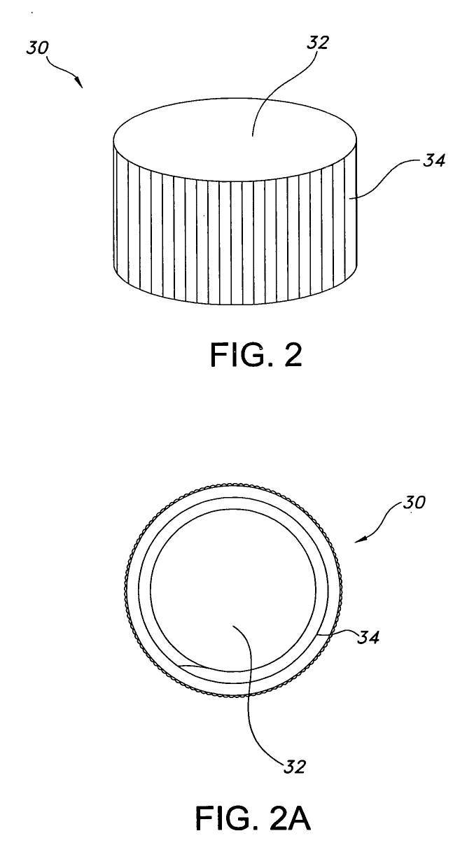

Referring now to the drawings in which reference characters refer to like parts throughout, FIG. 1 and FIG. 1A show a generally cylindrical coated tube suitable for growing and detecting oral bacteria. Tube 10 includes a cylindrical wall 12 which extends from an open end 14 to a closed end 16. Cells and culture fluids may be introduced into the body of tube 10 through open end 14. Tube 10 also includes an inner surface 18 and an opposed outer surface 20. Inner surface 18 is coated with an agar coating 22, which is selective for gram positive bacteria. The neck 24 of tube 10 may be externally screw-threaded for receipt of a cap 30, which is further shown in FIG. 2 and FIG. 2A. Cap 30 is for closing open end 14 of tube 10. Cap 30 has a top wall 32 and a depending annular skirt 34 for screw attachment to open end 14 of tube 10.

As can be seen in FIG. 1 and FIG. 1A, in one embodiment, tube 10 is only partially coated with an agar coating selective for gram positive bacteria. For example, in FIG. 1 and FIG. 1A, the closed end 16 of tube 10 remains substantially uncoated. However, embodiments are also possible where the entire inner surface of tube 10 is coated with an agar coating selective for gram positive bacteria.

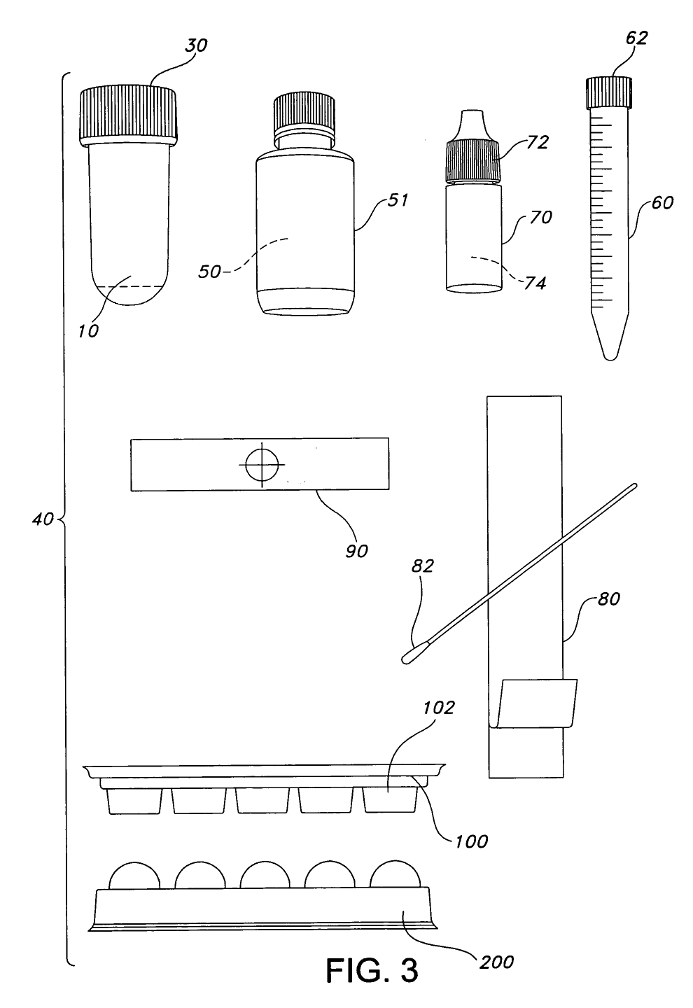

Referring now to FIG. 3, one embodiment of a kit 40 according to the present invention is shown. Kit 40 at least includes the following components: coated tube 10, the inner surface of which is coated with an agar coating selective for oral bacteria; screw cap 30 for closing open end 14 of tube 10; and liquid media 50. Liquid media 50 may be provided in container 51 and is sterile. The kit components are desirably stored at room temperature.

In desired embodiments, kit 40 further includes an uncoated, sterile tube 60, which can be at least partially filled with media 50 prior to inoculation of the media with an oral sample from a tooth surface. Tube 60 is preferably a sterile, transparent plastic tube having a screw cap 62.

The test kit 40 also preferably includes a container 70 which has a screw cap 72 thereon, and which contains therein an antibiotic reagent 74. Antibiotic reagent 74 prevents the growth of microorganisms which might otherwise interfere with the inventive method for detecting oral bacteria.

Also preferably included with the kit is a sterile package 80, which contains an absorbent material. In the embodiment shown in FIG. 3, the absorbent material is a swab 82 for obtaining oral samples from a tooth surface.

The test kit 40 may also include a counting strip 90 which can be placed around outer surface 20 of tube 10 in order to determine the number of oral bacterial colonies within a given target area on the tube. If desired, the colony count within the targeted area may be compared to a standard colony density comparison chart (not shown), which may also be included in the kit, in order to obtain a semi-quantitative determination of the bacteria.

Moreover, the test kit may include a holder 100 for the coated tube 10. In the embodiment shown in FIG. 3, the holder is a tray. In some embodiments, the kit may further include a lid 200 for holder 100. The holder 100 would preferably include at least one hole 102 for receiving the coated tube. In desired embodiments, the holder preferably includes multiple holes for receiving multiple tubes. As will be described in further detail below, coated tube 10 is preferably incubated in the cap-down position at 37° C. overnight in an incubator during the detection method of this invention. If desired, the tube may be placed in holder 100 during this incubation. The kit can also include a paper insert containing the directions for use of the kit.

Preferably, all of the kit components of FIG. 3 are contained within foil packaging (not shown). The foil packaging minimizes moisture loss.

The inventive method for the detection of oral bacteria involves taking a test sample for the surfaces of the teeth from a patient. For example, in one embodiment, the dentist removes a sterile swab from it's packaging and aseptically collects a patient's specimen by swabbing the patient's teeth. In one example, the patient's teeth are swabbed with a back-and-forth motion five times on both the inner and outer surfaces of the teeth. Once a sample or specimen is obtained from the patient using a swab or other equivalent device, the swab or other equivalent device having the patient sample or specimen is inserted into tube 60 containing liquid media 50 in order to inoculate the media. The sample may be squeezed from the swab by gently pressing the swab against the walls of the tube that contains the liquid media. The swab is then removed from the container, and the liquid is mixed to ensure proper dispersal of the sample within the media. A cap is then placed on tube 60 including the patient's specimen.

Once the specimen has been collected, the agar coated tube of the present invention may be readied for receipt of the specimen. In particular, a predetermined amount of a liquid antibiotic reagent 74, such as a liquid bacitracin composition, may be added to the coated tube. Thereafter, the coated tube 10 is capped using cap 30 and the antibiotic is dispersed into the agar coating by rotating the tube slowly so as to create a uniform coating of the antibiotic. The media, which has been inoculated with the patient's specimen, is then transferred into the agar coated tube 10 containing the dispersed antibiotic. Thereafter, the coated tube is capped using cap 30 and the contents are mixed by inversion. Subsequently, the liquid contents of the coated tube are removed. The tube is then incubated until bacterial colonies are detectable on the agar coating. For example, in some embodiments, the tube is incubated at 37° C. In some further embodiments, the tube is incubated for about 15 to about 24 hours.

After incubation, the growth density of the oral bacteria on the agar coating may be evaluated. Any viable colonies within a given target area on the agar coating may be counted. For example, a counting strip 90 may be placed around the coated tube 10, and the colony count may be determined within the target area. Preferably, the colony count is taken from at least three different areas on the tube, and the results are averaged.

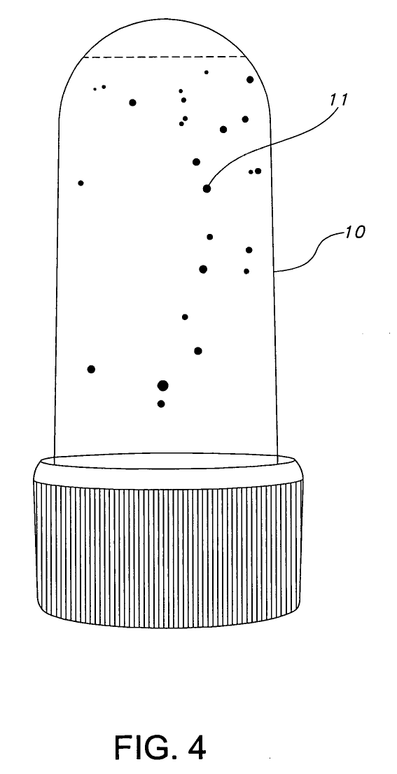

FIG. 4 shows the coated cell culture tube 10 after it has been brought in contact with a patient's oral specimen and thereafter incubated for overnight at 37° C. As can be seen, coated tube 10 includes bacterial colonies 11 on inner surface 18. As described above, the number of colonies within a given target area(s) on the tube may be counted. If desired, the dentist or other dental professional may compare the colony density on the tube with a standard comparison chart in order to determine the colony density of the patient's oral bacteria.

In some preferred embodiments, the tube which is coated is made of a substantially clear plastic, and the agar coating includes an indicator dye, such as Trypan Blue. This permits the colonies to appear clearly as dark blue colonies on a lighter blue background. Another example of an indicator dye is phenolsulfonphthalein. Indicator dyes may be associated with a measurable property change, such as color or pH. For example, a measurable property change may be the appearance of a pigment as a color change indicating the presence of a particular type of oral bacteria.

As described above, the device of the present invention is preferably a generally cylindrical clear plastic tube, the inner surface of which is coated with an agar coating. In some embodiments, the substantially clear plastic material is a polyolefin. In some embodiments, the polyolefin is polystyrene.

The coated tube can vary in size, but is generally a clear plastic tube which is capable of holding between 10 to 50 ml of liquid media, and includes a screw cap. The cap is preferably designed to maintain sterile conditions prior to addition of the patient's specimen. During culturing, the cap minimizes moisture loss and protects the agar from bacterial contamination from the environment.

The agar coating on the inner surface of the tube of the present invention is preferably a spun agar coating. For example, the inner surface of a tube may be coated using a spin-coater, which disperses the liquid solution of the agar medium into the tube and then agar-coats the tube during centrifugation. Spin-coater/chillers are well known in the art. The agar-coated tubes may be stored at room temperature until use.

As shown in the figures, the agar coating need not cover the entire inner surface of the tube. However, it is preferred that the inner surface be at least substantially coated with an agar coating selective for gram positive bacteria. In one embodiment, the agar coating is selective for mutans streptococci.

In one embodiment, the agar coating includes a Mitis Salivarius agar medium, which has been enriched with sucrose. Mitis Salivarius agar is a term well known in the art. Chapman investigated methods for isolating streptococci and formulated Mitis Salivarius agar (J. Bacteriol. 48:113, 1994; Am. J. Dig. Dis. 13:105, 1946; Trans. N.Y. Acad. Sci. (Series 2) 10:45, 1947). Streptococcus mutans can be distinguished by its colonial morphology on Mitis Salivarius agar. Mitis Salivarius agar contains peptones as sources of carbon, nitrogen, vitamins and minerals. Sucrose and dextrose are carbohydrate sources. Dipotassium Phosphate is the buffering agent. Trypan Blue is absorbed by the colonies, producing a blue color. Crystal violet and Potassium Tellurite inhibit most gram-negative bacilli and gram-positive bacteria except streptococci. Agar is the solidifying agent. The conventional formulation for Mitis Salivarius agar per liter is shown below in Table 1.

TABLE 1
Unmodified Mitis Salivarius Agar Formulation/1 Liter

| | | |
|---|---|---|
| Enzymatic Digest of Casein | 15 | g |
| Enzymatic Digest of Animal Tissue | 5 | g |
| Sucrose | 50 | g |
| Dextrose | 1 | g |
| Dipotassium Phosphate | 4 | g |
| Trypan Blue | 0.075 | g |
| Crystal Blue | 0.0008 | g |
| Agar | 15 | g |
| 1% Potassium Tellurite | 1 | ml |

Whereas the conventional Mitis Salivarius agar formulation may be employed as the coating composition, the present inventor has also employed modified Mitis Salivarius agar coating compositions, which have been found to be more selective for the detection of streptococcus mutans and to produce better results, as compared to the conventional formulation shown in Table 1. These modified compositions will be described in greater detail below.

In some embodiments, the agar is desirably present in the coating in an amount of about 1.5% (w/v). Moreover, in some embodiments, the agar coating includes sucrose as a selective saccharide in an amount of about 10 to about 20% (w/v).

The agar coating desirably includes at least one peptone. Suitable peptones include meat and casein peptones. These peptones compensate for any nutritional loss in the media due to sterilization of the media. In some embodiments, the at least one peptone is present in the agar coating in an amount of about 0.1 to about 10% by weight.

Selective agents may be present in the agar coating. These selective agents include potassium tellurite. In some embodiments, the potassium tellurite is present in the agar coating in an amount of about 0.001 to about 1% by weight.

The agar coating may also include other components. For example, the agar coating can include stabilizing agents, buffering agents, as well as indicator dyes. Such agents are well known in the art. For example, pH adjustment additives may include water-soluble phosphate salts. Moreover, a base may be added so as to adjust the pH to a higher alkalinity. The pH of the agar coating is preferably about 7 to about 7.5. Stabilizing agents can include such agents as ammonium sulfate. Moreover, indicator dyes can include such agents as Trypan Blue or phenolsulfonphthalein. Trypan Blue gives the colonies a dark blue color against a lighter blue background.

In some embodiments, the agar coating may include an antibiotic. For example, the antibiotic may be bacitracin, which is a polypeptide. In some embodiments, the bacitracin is present in an amount of about 0.1 units/ml to about 2 units/ml. In one desired embodiment, bacitracin is present in an amount of about 0.2 units/ml. In some embodiments, it may be desirable to disperse the antibiotic into the gel coating just prior to culturing so as to avoid any stability problems that may be associated with the antibiotic.

As described above, the present invention provides a liquid media suitable for transferring bacteria into the cell culture tube of the present invention. As further described below, this media is inoculated with a patient's specimen. The inoculated media is transferred into the inventive culture tube once the culture tube has been readied for receipt of the specimen.

The present inventor has found that the particular liquid media used for inoculation and transfer of the patient's oral bacteria into the coated culture tube has a pronounced effect on the outcome of the detection method of the present invention. For example, it was found that formulations such as phosphate buffer saline, were not supportive of oral bacteria. Also, the inclusion of a polyol, such as mannitol, in sufficient amounts, made the method of the present invention more selective for the detection of oral bacteria, such as streptococcus mutans.

In some embodiments, the liquid media used to transfer the bacteria into the inventive culture tube includes: at least one saccharide; at least one peptone; and at least one polyol. These components are present in sufficient amounts to be supportive of the oral bacteria.

In some embodiments, the liquid media includes at least one polyol in an amount of about 0.1 to about 5% (w/v). In some preferred embodiments, the liquid media includes at least one polyol in an amount of about 0.1 to about 0.25% (w/v). An example of a suitable polyol is mannitol.

In further embodiments, the liquid media includes at least one peptone in an amount of about 0.1 to about 5% (w/v). In some preferred embodiments, the at least one peptone is present in the liquid media in an amount of about 0.1 to about 0.5% (w/v).

In other embodiments, the liquid media includes at least one saccharide, such as sucrose. The sucrose may be present in the liquid media in an amount of about 0. 1 to about 5% (w/v). In some preferred embodiments, the sucrose is present in the liquid media in an amount of about 0.1 to about 0.5% (w/v).

In some further embodiments, the liquid media composition includes: at least one peptone in an amount of 0.1 to about 0.5% (w/v); at least one saccharide compound in an amount of about 0.1 to about 0.5% (w/v); and at least one polyol in an amount of about 0.1 to about 0.25% (w/v). As described above, the saccharide may be sucrose. However, other saccharides are suitable, such as fructose or glucose. Also, combinations of saccharides are possible. Moreover, as described above, mannitol is one example of a useful polyol to include in the liquid transfer media. However, other polyols are suitable. These include, for example, sorbitol, maltitrol, lactitol, etc. Combinations of the polyols are also possible.

### EXAMPLES

### Example 1

### Preparation of Modified Mitus Salivarius Agar Coating Composition A

A conventional formulation of Mitis Salivarius Agar Medium is prepared in the present example using Mitis Salivarius Agar from Difco, except that it was modified by adding additional sucrose and by adding bacitracin, such that the final concentration of sucrose in the coating composition was 15%, and the final concentration of bacitracin was 0.2 units/ml. This agar medium may be used as a coating on the inner surface of the device of the present invention.

As shown in Table 1 above, in a conventional Mitis Salivarius agar formulation, sucrose is incorporated in a 5% concentration, and is utilized only as an energy source. However, the present inventor has found that, when incorporated in higher amounts (e.g., 15-20% final sucrose), the additional sucrose serves as an effective humectant that substantially prevents bacterial colonies from “running into each other” as would otherwise occur under the wet conditions in the tube.

Approximately 90 g of Difco™ Mitis Salivarius Agar and 100 g of additional sucrose were suspended in 1 liter of purified water, and mixed thoroughly. Thereafter, the mixture was heated by agitation, and boiled for one minute to completely dissolve the powder. The mixture was then autoclaved at 121° C. for 15 minutes. Subsequently, the sterile medium was cooled to 50-60° C., and 1 ml of a 1% filter sterilized potassium tellurite solution was added. The resulting medium was dispensed into sterile tubes, and the tubes were coated with the agarose medium using a spin-coater, as described below in Example 3. Bacitracin was added to the coated tubes just prior to use, as described in Example 4 below.

### Example 2

### Preparation of Modified Mitis Salivarius Agar Coating Composition B

The present example is directed to the preparation of another modified Mitis Salivarius agar medium. This agar medium may be used as a coating on the inner surface of the device of the present invention. Table 2 below shows the components of this modified formulation per liter of purified water.

TABLE 2
Modified Mitis Salivarius Agar Composition B (per liter)
Part A

| | | |
|---|---|---|
| L-Cystine-HCL | 0.2 | g |
| Sodium Sulfite | 0.1 | g |
| sodium phosphate, dibasic | 1 | g |
| sodium bicarbonate | 2 | g |
| sodium acetate | 20 | g |
| Bacitracin | 200 | units |

Part B

| | | |
|---|---|---|
| Peptone, bacteriological | 15 | g |
| Yeast Extract, bacteriological | 5 | g |
| sucrose | 200 | g |
| Agar, bacteriological | 15 | g |

Part C

| | | |
|---|---|---|
| Trypan Blue | 0.075 | g |
| 1% Potassium Tellurite | 1 | ml |
| Crystal Violet | 0.0008 | g |

Part A components, all soluble salts, were dissolved in 100 ml water. The pH was adjusted to pH 7.83 at 25° C. by addition of sodium hydroxide, and then sterile filtered through a 0.1 μ filter.

Part B components were suspended in 800 ml water, then heated with frequent agitation, and boiled for one minute to completely dissolve the medium. Thereafter, the composition was autoclaved at 121° C. for 30 minutes. Subsequently, the sterile, autoclaved medium was cooled to 50-60° C., and the Part A salts (sterile filtered) were added to the medium. The volume was then adjusted to 1 L with sterilized water.

Optionally, one or more of the Part C components (Mitis Salivarius specific additives used as growth inhibitors) are added to either Part A or Part B to enhance detection of streptococcal mutans.

In the present embodiment, the Trypan Blue component was added to the medium.

The resulting medium was held at 50-60° C., and then was dispensed into sterile tubes. The tubes were coated with the agar medium using a spin-coater, as described below in Example 3. Bacitracin was added to the coated tubes just prior to use, as described in Example 4 below.

### Example 3

### Spin-Coating Procedure

The present example describes the procedure used to prepare a coated tube of the present invention. A modified Mitus Salivarius medium was prepared according to Example 1 or 2. The resulting sterile medium was equilibrated to about 50-60° C. in a water bath located near a spin-coater. Between 2.6-2.7 ml of the sterile agar medium were dispensed into each sterile tube, the tubes capped so as to maintain sterile conditions, and the tubes were coated using a spin-coater/chiller. In particular, the tubes were spun at 2300 rpm at −8° C. for 48 seconds to obtain a gel coating on the inner surface of the tubes. The coated tubes were stored at room temperature until use.

### Example 4

### Method of Detecting Mutans Streptococci

The present example is directed to a method for detecting mutans streptococci on surfaces of teeth. A sterile swab was removed from it's packaging and used to aseptically collect a patient's specimen by swabbing the patient's teeth. The patient's teeth were swabbed with a back-and-forth motion five times on both the inner and outer surfaces of the teeth. Once a sample or specimen was obtained from the patient using the swab, the swab containing the sample was inserted into a tube containing liquid media in order to inoculate the media. The liquid media for inoculation included the components shown below in Table 3, which are suspended in one liter of distilled water.

TABLE 3
Components of Liquid Media per Liter (w/v)

| | |
|---|---|
| Peptone | 5 g |
| sucrose | 5 g |
| mannitol | 2.5 g |

The sample was squeezed from the swab by gently pressing the swab against the walls of the tube that contains the liquid media. The swab was then discarded and the liquid was mixed to ensure proper dispersal of the sample within the media. A cap was then placed on the coated tube including the patient's specimen.

Once the specimen was collected, the agar coated tube of the present invention was readied for receipt of the specimen. In particular, a predetermined amount of a liquid bacitracin composition was added to the coated tube, such that the final concentration was about 0.2 units/ml. The agar coated tube was capped and the antibiotic was dispersed into the agar coating by rotating the tube slowly so as to create a uniform film coating of the antibiotic. The transfer media, which was inoculated with the patient's specimen, was then transferred into the agar coated tube containing the dispersed antibiotic. Thereafter, the agar coated tube was capped and the contents were mixed by inversion. Subsequently, the liquid contents of the coated tube were disposed of. The tube was recapped and then incubated overnight at 37° C. After incubation, the tube was inspected for the presence of detectable bacterial colonies. The growth density of the oral bacteria on the agar coating was evaluated. Any colonies within a given target area on the agar coating were counted. A counting strip was placed around the coated tube, and the colony count was determined within a target area. Thereafter, the colony count was taken from at least two other areas on the tube, and the results were averaged.

### Example 5

### Comparison of the Coated Device of the Present Invention With a Prior Device

In the present example, the performance of a kit according to the present invention was compared to that of a commercial caries risk test, Vivacare line CRT (Caries Risk Test), which is available from Vivadent, Liechtenstein, Europe. The prior kit is comprised of a slide attached to the cover of a vial. One side of the slide is coated with a Mitis Salivarius agar enriched with sucrose for the cultivation of mutans streptococci, while the medium on the other side (Rogosa agar) was for the cultivation of lactobacilli.

Samples were obtained from twenty five people selected randomly. For the CRT kit test, salivary samples were collected and tested in accordance with the manufacturer's instructions. The CRT-tested samples were incubated at 37° C. for 48 hours. For the inventive kit test, samples were collected from teeth surfaces and tested in accordance with the method described above in Example 4. The results are shown below in Table 4, where the growth density of mutans streptococci was evaluated, and then scored by a low/moderate/high classification depending on if very few colonies were colonies were detected, moderate amounts of colonies were detected, or high amounts of colonies were detected, respectively. Bacterial growth was expressed as colony forming units (cfu). The classification system provided for a classification of the patients into patients of low, moderate or high dental caries risk. A determination of a high risk patient enables preventative measures to be taken against dental caries, such as professional teeth cleaning, fluoride treatment, antibiotics, or anti-bacterial therapy with agents such as chlorhexidine.

TABLE 4
Comparison of Results Obtained From Prior Test Kit vs. Inventive Kit

| Sample | Inventive Kit | | CRT kit | |
|---|---|---|---|---|
| No. | average cfu | classification* | classification* | Correlation |
| 1 | 2.6 | low | low | + |
| 2 | 30.0 | mod | mod | + |
| 3 | 15.0 | low | low | + |
| 4 | 46.0 | mod | high | +/− |
| 5 | 24.0 | mod | low | +/− |
| 6 | 19.0 | low | low | + |
| 7 | 56.0 | high | high | + |
| 8 | 15.3 | low | mod. | +/− |
| 9 | 128.0 | high | mod/high | +/− |
| 10 | 55.0 | high | high | + |
| 11 | 63.0 | high | low | − |
| 12 | 7.6 | low | low | + |
| 13 | 32.6 | mod | low | +/− |
| 14 | 43.0 | mod | low/mod | +/− |
| 15 | 29.0 | mod | low/mod | +/− |
| 16 | 36.0 | mod | mod | + |
| 17 | 5.6 | low | low | + |
| 18 | 15.0 | low | low | + |
| 19 | 43.3 | mod | high | +/− |
| 20 | 148.0 | high | mod | +/− |
| 21 | 29.3 | mod | low | +/− |
| 22 | 89.3 | high | high | + |
| 23 | 2.3 | low | low | + |
| 24 | 7.3 | low | low | + |
| 25 | 2.6 | low | low | + |

*For the classification, mutans streptococci growth was scored as follows: low, ≦20 cfu; moderate, >20 cfu, but <50 cfu; or high, ≧50 cfu.

Based on the results shown in Table 4, it can be seen that fourteen out of the twenty five samples gave consistent results (+) between the inventive kit test and the CRT test kit. In ten of the samples, the correlation was close, or off by up to one classification (+/−). The results of one of the samples (sample No. 11) differed significantly between the inventive and CRT test (−) in that the inventive test demonstrated a high cfu count, whereas the CRT test demonstrated a low cfu count. A likely explanation for this variation is the difference between the sampling methods. In particular, the inventive kit utilizes samples taken directly from the teeth surfaces, whereas the CRT kit uses salivary samples.

Although the test results correlated well, for the most part, between the inventive and CRT tests, the CRT test presented several disadvantages. For example, the CRT test kit method involves a 48 hour incubation before the colony density on the slide can be evaluated. It is also an invasive and time consuming method for the patient. For example, the patient is asked to chew paraffin wax for several minutes during which time saliva needs to be collected at regular time intervals. Furthermore, the agar coating on the CRT slide is easily scratched by the pipette used to transfer the salivary sample onto the slide. For example, in two of the twenty five CRT tests, the agar coatings on the slides were accidentally scratched in this way, making the results difficult to read. Moreover, the CRT slides are opaque, and counting required a backlight to better view the colonies. Also, for two of the twenty five samples, it was difficult to tell if the CRT slides had been completely saturated with the saliva samples. Furthermore, it was not possible to place a counting grid directly against the slides when evaluating for bacterial growth, making the counting less accurate. Moreover, the CRT test does not take samples directly from the teeth surfaces, where mutans streptococci reside as a major component of dental plaques. Therefore, in certain samples (e.g., sample 11), the counts from the salivary samples may be lower than would otherwise be obtained if the sample was taken from teeth surfaces. The consequence of this is that certain high risk patients may go undetected using the CRT kit.

In contrast, the inventive test method involves half the incubation time of the CRT test method (i.e., 24 hrs., as opposed to 48 hrs.). Also, the present method is not time consuming or invasive for the patient. Moreover, the agar coating on the inner surface of the device of the present invention is not damageable during transfer of the sample into the tube. Also, the coated tube of the present invention is transparent, instead of opaque like the CRT slide. This transparency makes the bacterial colonies easier to see and count. Also, a counting grid may be placed directly against the outer surface of the transparent tube during counting. This makes the counting procedure more accurate than that for the CRT kit. Furthermore, the inventive kit utilizes samples taken directly from the teeth surfaces, where mutans streptococci reside as a major component of dental plaques.

What is claimed is:

1. A device for detecting oral bacteria comprising:
a generally cylindrical tube having an open end for receiving a sample and an opposed closed end, said tube comprising an inner surface and an opposed outer surface, said inner surface being coated with an agar coating selective for gram positive bacteria.

2. The device of claim 1, further comprising a cap for closing the open end of said tube, said cap having a top wall and a depending annular skirt for screw attachment to said open end of said tube.

3. The device of claim 1, wherein the tube is substantially clear.

4. The device of claim 1, wherein the tube is made from a substantially clear plastic material.

5. The device of claim 1, wherein the substantially clear plastic material is a polyolefin.

6. The device of claim 5, wherein the polyolefin is polystyrene.

7. The device of claim 1, wherein the agar coating is a spun agar coating.

8. The device of claim 1, wherein the agar coating is selective for mutans streptococci.

9. The device of claim 1, wherein the agar coating comprises Mitis Salivarius agar enriched with sucrose.

10. The device of claim 1, wherein the agar coating comprises agar in an amount of about 1.5% (w/v).

11. The device of claim 1, wherein the agar coating comprises sucrose in an amount of about 10 to about 20% (w/v).

12. The device of claim 1, wherein the agar coating comprises an antibiotic.

13. The device of claim 12, wherein the antibiotic is bacitracin.

14. The device of claim 1, wherein the agar coating comprises at least one peptone.

15. The device of claim 14, wherein the at least one peptone is present in the agar coating in an amount of about 0.1 to about 10% (w/v).

16. The device of claim 1, wherein the agar coating comprises potassium tellurite.

17. The device of claim 16, wherein the potassium tellurite is present in the agar coating in an amount of about 0.001 to about 1% (w/v).

18. The device of claim 1, wherein the agar coating comprises yeast extract.

19. The device of claim 18, wherein the yeast extract is present in the agar coating in an amount of about 0.5% (w/v).

20. The device of claim 1, wherein the agar coating comprises at least member of the group consisting of stabilizing agents, buffering agents and indicator dyes.

21. A method for detecting oral bacteria comprising:
providing an oral sample from a tooth surface;
providing a generally cylindrical tube having an open end and an opposed closed end, said tube comprising an inner surface and an opposed outer surface, said inner surface being coated with an agar coating selective for oral bacteria;
inoculating liquid media with the oral sample;
transferring the inoculated media into the tube;
removing the inoculated media from the tube;
subsequently incubating the tube until bacterial colonies are detectable on the agar coating.

22. The method of claim 21, further comprising evaluating the growth density of the oral bacteria on the agar coating.

23. The method of claim 21, further comprising counting the bacterial colonies on the agar coating.

24. The method of claim 21, further comprising dispersing an antibiotic into the agar coating before the transferring step.

25. The method of claim 21, wherein the tube is incubated at a temperature of about 37° C.

26. The method of claim 21, wherein the tube is incubated for about 15 to about 24 hours.

27. The method of claim 21, wherein the agar coating is a spun agar coating.

28. The method of claim 21, wherein the agar coating is selective for gram positive bacteria.

29. The method of claim 21, wherein the agar coating is selective for mutans streptococci.

30. The method of claim 21, wherein the agar coating comprises Mitis Salivarius agar enriched with sucrose.

31. The method of claim 21, wherein the agar coating comprises sucrose in an amount of about 10 to about 20% (w/v).

32. The method of claim 21, wherein the agar coating comprises Mitis Salivarius agar in an amount of about 1.5% (w/v).

33. The method of claim 21, wherein the agar coating comprises an antibiotic.

34. The method of claim 33, wherein the antibiotic is bacitracin.

35. The method of claim 21, wherein the agar coating comprises at least one peptone.

36. The method of claim 35, wherein the at least one peptone is present in the agar coating in an amount of about 0.1 to about 10% (w/v).

37. The method of claim 21, wherein the agar coating comprises potassium tellurite.

38. The method of claim 37, wherein the potassium tellurite is present in the agar coating in an amount of about 0.001 to about 1% (w/v).

39. The method of claim 21, wherein the agar coating comprises at least member of the group consisting of stabilizing agents, buffering agents and indicator dyes.

40. The method of claim 21, wherein the liquid media is selective for gram positive bacteria.

41. The method of claim 21, wherein the liquid media is selective for mutans streptococci.

42. The method of claim 21, wherein the liquid media comprises at least one polyol in an amount of about 0.1 to about 5% (w/v).

43. The method of claim 42, wherein the liquid media comprises at least one polyol in an amount of about 0.1 to about 0.25% (w/v).

44. The method of claim 21, wherein the liquid media comprises at least one peptone in an amount of about 0.1 to about 5% (w/v).

45. The method of claim 44, wherein the liquid media comprises at least one peptone in an amount of about 0.1 to about 0.5% (w/v).

46. The method of claim 21, wherein the liquid media comprises sucrose in an amount of about 0.1 to about 5% (w/v).

47. The method of claim 46, wherein the liquid media comprises sucrose in an amount of about 0.1 to about 0.5% (w/v).

48. The method of claim 21, wherein the liquid media comprises:
at least one peptone in an amount of about 0.1 to about 0.5% (w/v);
at least one saccharide compound in an amount of about 0.1 to about 0.5% (w/v); and
at least one polyol in an amount of about 0.1 to about 0.25% (w/v).

49. A kit for detecting oral bacteria comprising:
a generally cylindrical tube having an open end for receiving a sample and an opposed closed end, said tube comprising an inner surface and an opposed outer surface, said inner surface being coated with an agar coating selective for oral bacteria;
a cap for closing the open end of said tube;
liquid media.

50. The kit of claim 49, wherein the agar coating is a spun agar coating.

51. The kit of claim 49, wherein the agar coating is selective for gram positive bacteria.

52. The kit of claim 49, wherein the agar coating is selective for mutans streptococci.

53. The kit of claim 49, wherein the agar coating comprises Mitis Salivarius agar enriched with sucrose.

54. The kit of claim 49, wherein the agar coating comprises sucrose in an amount of about 10 to about 20% (w/v).

55. The kit of claim 49, wherein the agar coating comprises agar in an amount of about 1.5% (w/v).

56. The kit of claim 49, wherein the agar coating comprises at least one peptone.

57. The kit of claim 56, wherein the at least one peptone is present in the agar coating in an amount of about 0.1 to about 10% (w/v).

58. The kit of claim 49, wherein the agar coating comprises potassium tellurite.

59. The kit of claim 58, wherein the potassium tellurite is present in the agar coating in an amount of about 0.001 to about 1% (w/v).

60. The kit of claim 49, wherein the agar coating comprises at least member of the group consisting of stabilizing agents, buffering agents and indicator dyes.

61. The kit of claim 49, wherein the liquid media is selective for gram positive bacteria.

62. The kit of claim 49, wherein the liquid media is selective for mutans streptococci.

63. The kit of claim 49, wherein the liquid media comprises at least one polyol in an amount of about 0.1 to about 0.25% (w/v).

64. The kit of claim 63, wherein the polyol is mannitol.

65. The kit of claim 49, wherein the liquid media comprises:
at least one peptone in an amount of about 0.1 to about 0.5% (w/v);
at least one saccharide in an amount of about 0.1 to about 0.5% (w/v); and
at least one polyol in an amount of about 0.1 to about 0.25% (w/v).

66. The kit of claim 65, wherein the saccharide is sucrose; and the polyol is mannitol.

67. The kit of claim 49, further comprising an antibiotic reagent.

68. The kit of claim 67, wherein the antibiotic reagent is bacitracin.

69. The kit of claim 49, further comprising absorbent material for obtaining an oral sample from a tooth surface.

70. The kit of claim 69, wherein the absorbent material is a swab.

71. The kit of claim 49, further comprising a holder for the coated tube.

72. The kit of claim 71, wherein the holder is a tray with at least one hole for receiving the coated tube.

73. The kit of claim 49, further comprising a substantially clear grid for counting bacterial colonies.

74. The kit of claim 49, further comprising an uncoated sterile tube.

75. A cell culture tube comprising an inner surface substantially coated with a composition selective for oral bacteria, the composition comprising Mitis Salivarius agar enriched with sucrose, the sucrose being present in an amount of about 10 to about 20% (w/v).

76. The cell culture tube of claim 75, wherein the coated tube is made of a substantially clear plastic material.

77. A sterile liquid transfer media suitable for bacterial transport into the device of claim 1 comprising:
at least one peptone in an amount of about 0.1 to about 0.5% (w/v);
at least one saccharide in an amount of about 0.1 to about 0.5% (w/v); and
at least one polyol in an amount of about 0.1 to about 0.25% (w/v).

78. The liquid transfer media of claim 77, wherein the saccharide is sucrose; and the polyol is mannitol.
